(19) United States
(12) Patent Application Publication
(10) Pub. No.: US 2023/0374614 A1
(43) Pub. Date: Nov. 23, 2023

(54) PRIMERS, KIT AND METHOD FOR DETECTING OF AFRICAN SWINE FEVER VIRUS

(71) Applicant: ARMY MEDICAL UNIVERSITY, CHONGQING (CN)

(72) Inventors: Jintao Li, CHONGQING (CN); Xiaoyan Ding, CHONGQING (CN); Jiuxiang He, CHONGQING (CN); Yuxin Zhou, CHONGQING (CN); Xiaoyang Zhou, CHONGQING (CN)

(21) Appl. No.: 18/317,421

(22) Filed: May 15, 2023

(30) Foreign Application Priority Data
May 17, 2022 (CN) 202210536550X

(51) Int. Cl. C12Q 1/70

(52) U.S. Cl. CPC C12Q 1/701; C12Q 2600/166

(57) ABSTRACT

Primers, a kit and a method for detecting African swine fever virus are provided, which relates to virus detection technologies. The primers are used to detect vp72 gene of African swine fever virus, and nucleotide sequences thereof are shown in SEQ ID NO: 6 and SEQ ID NO: 7. The kit includes the primers. The method uses the primers or the kit to perform dye fluorescence quantitative polymerase chain reaction (PCR) on genomic DNA of a sample to be detected. This method has strong specificity, high sensitivity, good reproducibility, and lower cost than the TaqMan probe method.

### TECHNICAL FIELD

The disclosure relates to the field of virus detection technologies, and more particularly to primers, a kit and a method for detecting of African swine fever virus (AFSV).

### STATEMENT REGARDING SEQUENCE LISTING

The sequence listing associated with this application is provided in text format in lieu of a paper copy and is hereby incorporated by reference into the specification. The name of the XML file containing the sequence listing is 23051TBYX-USP1-SL.xml. The XML file is 12,834 bytes; is created on May 15, 2023; and is being submitted electronically via EFS-Web.

### BACKGROUND

African swine fever virus (ASFV) is a double-stranded DNA virus, which contains five layers of structure, including envelope, capsid, bilayer inner membrane, core shell and genome, with more than 30,000 protein subunits (Wang et al., 2019), replicates mainly in granulocytes, especially antigen-presenting cells (APCs), such as monocytes/macrophages and dendritic cells, causing severe necrosis, hemorrhage, and eventually death of tissues (Malmquist&Hay, 1960; Wardley&Wilkinson, 1977; Mebus, 1988; Tulman&Rock, 2001; Sdnchez-Codón et al., 2018).

African swine fever (ASF) caused by this virus first occurred in the African country of Kenya in 1921, was introduced in Europe in 1957, in the Americas in 1971, and subsequently became a local epidemic disease in Sardinia in the Mediterranean in 1982. By 2007, the disease was introduced to some countries in Eastern Europe (Sdnchez Codón et al., 2018); in 2018, it was introduced to China (Zhao et al., 2019), sweeping across four continents: Europe, America, Asia, and Africa, causing an estimated global economic loss of 2 billion dollars. The disease is characterized by acute onset, and rapid transmission, with morbidity and mortality rates reaching 100%, and no effective vaccine is available at present. Once the infection is confirmed, animals are immediately culled to prevent the further spread and spread of ASFV. Therefore, rapid and reliable detection of ASFV is a crucial emergency control measure.

In the early diagnosis of ASFV, polymerase chain reaction (PCR) method is dominate. In 2016, Luo et al. successfully designed a pair of highly sensitive and universal PCR primers and established an improved traditional PCR detection method by comparing the vp72 gene coding sequences of 35 ASFV strains (Luo et al., 2016). However, this method is time-consuming, easily contaminated, and requires electrophoresis detection. Fluorescence quantitative PCR combines fluorescence detection with traditional PCR, which has the advantages of simple operation, high sensitivity, strong specificity, and good reproducibility. There are two types of fluorescence quantitative PCR methods: SYBR Green dye method and TaqMan probe method. Comparatively, the SYBR Green dye method is more economical because it does not require the synthesis of probes with fluorescent groups, and its specificity mainly depends on the preferred primer pairs, while the TaqMan probe method, which requires the synthesis of specific probes, is more expensive but has better specificity than the SYBR Green dye method. The diagnostic criteria for ASFV established by the World Organization for Animal Health (OIE) use a quantitative polymerase chain reaction (qPCR) method based on the vp72 gene of the ASFV, which utilizes degenerate primers and has poor specificity (Ren, et al., 2020). Zhang Quan et al. established the TaqMan probe method for fluorescence quantitative PCR detection based on the vp72 gene, with a sensitivity of 20 copies per microliter abbreviated as copies/μL (Zhang Quan et al., 2007). The TaqMan probe method for the detection of ASFV based on the vp72 gene established by Ren Ming et al. and Li Hongli et al. was able to detect as low as 10 copies/μL, which was comparable to that of OIE primers (Ren et al., 2020 and Li Hongli et al., 2012). Therefore, it is necessary to establish a molecular detection method for ASFV that takes into account sensitivity, specificity and cost.

### SUMMARY

In order to establish an economic, rapid, accurate and sensitive molecular detection method for African swine fever virus (ASFV), specific primers are designed and screened from the conserved region of the vp72 gene of 30 ASFV strains by multiple sequence alignment, and a fluorescent dye quantitative polymerase chain reaction (qPCR) method based on the vp72 gene of ASFV is successfully established by optimizing the reaction system and reaction conditions. This method can detect 10 copies per microliter (copies/μL) positive plasmid standard of ASFV, has strong specificity and good reproducibility, and is more economical than the TaqMan probe method.

In an aspect, primers for detecting the vp72 gene of ASFV are provided, nucleotide sequences of the primers are as shown in SEQ ID NO: 6 and SEQ ID NO: 7.

In another aspect, a kit for detecting the vp72 gene of ASFV is provided, which includes the primers for detecting the vp72 gene of ASFV.

In an embodiment, the kit further includes a positive control and/or a negative control.

In an embodiment, the positive control is a standard plasmid containing part or all of the vp72 gene, and the negative control is a liquid used to dilute the positive control.

In an embodiment, the standard plasmid contains a nucleotide sequence as shown in SEQ ID NO: 1.

In an embodiment, the positive control is a standard plasmid solution with a concentration of 1×10² copies/μL.

In an embodiment, the kit further includes a polymerase chain reaction (PCR) premix; and the PCR premix includes a Taq enzyme (also referred to as Taq DNA polymerase), a deoxyribonucleotide triphosphate (dNTP) mixture, a TB Green dye, and a buffer.

In still another aspect, a fluorescence quantitative PCR method for detecting the vp72 gene of ASFV is provided, which includes performing the fluorescence quantitative PCR on a genome DNA of a sample to be detected through a dye method by using the primers or the kit.

In an embodiment, a reaction system for the fluorescence quantitative PCR includes 5 μL of 2×TB Green Premix Ex Taq II (Tli RNaseH Plus), 0.3 μL of 10 micromoles per liter (μM) upstream primer, 0.3 μL of 10 μM downstream primer, 1 μL of DNA Template, and 3.4 μL of double-distilled water (ddH₂O) without nuclease.

In an embodiment, reaction conditions for the fluorescence quantitative PCR include: pre-denaturation at 95 Celsius degree (° C.) for 30 seconds, denaturation at 95° C. for 5 seconds, annealing and extension at 60° C. for 30 seconds, 40 cycles.

It is proved through experiments that the fluorescence quantitative PCR detection method established by the disclosure has advantages such as high sensitivity, strong specificity, and good reproducibility. In the sensitivity test, a primer pair as shown in SEQ ID NO: 2 and SEQ ID NO: 3 (referred to as “primer pair P1” or “P1 primer pair” for short) that can specifically amplify the vp72 fragment of ASFV published in the literature is selected as a control, and the primer pair is more conservative than the PCR primer pair recommended by the World Organization for Animal Health (OIE) and has a higher sensitivity and a wider detection range. The results show that the P1 primer pair published in the literature shows good sensitivity in ordinary PCR, but the amplification efficiency is low in fluorescence quantitative PCR detection, with a detection limit of 10⁴ copies/μL (FIGS. 4-6), in this situation, it is not suitable for fluorescence quantitative PCR detection. The primer pair provided by the disclosure as shown in SEQ ID NO: 6 and SEQ ID NO: 7 (referred to as “primer pair P3” or “P3 primer pair”) has high sensitivity in fluorescence quantitative PCR detection, can detect 1 copy/μL of plasmid standard, and has good peak shape of dissolution curve and strong specificity (FIGS. 13-15). The reproducibility test results show that the intra-assay and inter-assay coefficients of variation of the vp72 gene of ASFV detected by the method of disclosure are less than 2% (Table 3), and the detection results are stable and reliable.

In conclusion, an economical, rapid, accurate and sensitive molecular detection method for ASFV is successfully established in the disclosure based on the selected amplification primers of vp72 gene. The method uses the SYBR Green dye method for detection, with strong specificity. The linear correlation R² of the standard curve can reach 0.9972, the sensitivity is higher than the TaqMan probe method published in the literature, the detection cost is lower than the TaqMan probe method, providing an economical and effective molecular diagnostic tool for the rapid diagnosis of ASFV.

### BRIEF DESCRIPTION OF DRAWINGS

FIG. 1 illustrates alignment results of a primer pair (P1) published in the literature with 30 complete coding sequences of vp72 genes of African swine fever virus (ASFV); where each dot (.) represents the same base; and positions of the primers are indicated by dashed boxes.

FIG. 2 illustrates alignment results of a primer pair (P2) designed by the disclosure with the 30 complete coding sequences of vp72 genes of ASFV; where each dot (.) represents the same base; and positions of the primers are indicated by a dashed box.

FIG. 3 illustrates alignment results of a primer pair (P3) designed by the disclosure with the 30 complete coding sequences of vp72 genes of ASFV; where each dot (.) represents the same base; and positions of the primers are indicated by dashed boxes.

FIG. 4 illustrates amplification curves of fluorescence quantitative polymerase chain reaction (PCR) detection using the P1 primer pair; where 10⁴, 10⁵, 10⁶, 10⁷, 10⁸, 10⁹, and 10¹⁰ represent copy numbers of plasmid standards.

FIG. 5 illustrates dissolution curves of fluorescence quantitative PCR detection using the P1 primer pair.

FIG. 6 illustrates standard curves of fluorescence quantitative PCR detection using the P1 primer pair.

FIG. 7 illustrates amplification curves of fluorescence quantitative PCR detection using the P2 primer pair; where 10¹, 10², 10³, 10⁴, 10⁵, 10⁶, 10⁷ represent copy numbers of plasmid standards.

FIG. 8 illustrates dissolution curves of fluorescence quantitative PCR detection using the P2 primer pair.

FIG. 9 illustrates standard curves of fluorescence quantitative PCR detection using the P2 primer pair.

FIG. 10 illustrates amplification curves of fluorescence quantitative PCR detection using the P3 primer pair; where 10¹, 10², 10³, 10⁴, 10⁵, 10⁶, and 10⁷ represent copy numbers of plasmid standards.

FIG. 11 illustrates dissolution curves of fluorescence quantitative PCR detection using the P3 primer pair.

FIG. 12 illustrates standard curves of fluorescence quantitative PCR detection using the P3 primer pair.

FIG. 13 illustrates optimized amplification curves of fluorescence quantitative PCR for the P3 primer pair; where 10⁰, 10¹, 10², 10³, 10⁴, 10⁵, and 10⁶ represent copy numbers of plasmid standards.

FIG. 14 illustrates optimized dissolution curves of fluorescence quantitative PCR for the P3 primer pair.

FIG. 15 illustrates optimized standard curves of fluorescence quantitative PCR for the P3 primer pair.

### DETAILED DESCRIPTION OF EMBODIMENTS

Technical solutions of the disclosure will be described in detail below with reference to specific embodiments and the accompanying drawings.

In the following embodiments, the blood used to extract genomic DNA of Bama Xiang pigs is taken from healthy Bama Xiang pigs which are not infected with African swine fever virus (ASFV). Bama Xiang pig is provided by Experimental Animal Center of Army Medical University.

Escherichia coli DH5a cells competent cells are purchased from Beijing Tsingke Xinye Biotechnology Co., Ltd.

PUC57 plasmid vector is a cloning vector purchased from Beijing Tsingke Xinye Biotechnology Co., Ltd. The vector size is 2710 base pairs (bp), the National Center for Biotechnology Information (NCBI) accession number of the vector sequence is Y14837.1, and the vector resistance is ampicillin.

Main reagents and consumable materials are as follows.

Real Time PCR fluorescent dye reagent TB Green® Premix Ex Taq™ II (Tli RNaseH Plus) is purchased from TAKARA Bio INC., article number (Art. No.) RR820Q. Plasmid DNA extraction kit is purchased from TIANGEN Biotech (Beijing) Co., Ltd, Art. No. DP103. Mini BEST Viral RNA/DNA Extraction Kit Ver. 5.0 kit is purchased from TAKARA Bio INC., Art. No. 9766. PrimeScript™ RT reagent Kit with gDNA Eraser kit is purchased from TAKARA Bio INC., Art. No. RR047A.

All primers are commissioned to be synthesized by BGI Genomics Co., Ltd. The works of gene synthesis, vector construction, and sequencing are commissioned to Beijing Tsingke Xinye Biotechnology Co., Ltd.

Unless otherwise specified, the reagents used in the following embodiments are all conventional reagents in the art, which can be purchased commercially or prepared according to conventional methods in the art, and the specifications are laboratory grade. Unless otherwise specified, the experimental methods and conditions used in the following embodiments are conventional experimental methods and conditions in the art and reference can be made to relevant experimental manuals, published literature, or manufacturer's instructions. Unless otherwise defined, all technical and scientific terms used herein have the same meanings as those skilled in the related art to which the disclosure belongs.

### Embodiment 1 Design and Synthesis of Primers for Fluorescence Quantitative Polymerase Chain Reaction (PCR) for ASFV

The full-length sequences of vp72 genes from 30 ASFV strains are selected on the NCBI website (Table 1), the conserved region of vp72 gene is screened by multiple sequence alignment through MegAlign software, and the nucleotide sequence of the conserved region is shown in SEQ ID NO: 1. Specific primers are designed and screened by using the NCBI Primer design tool in the conserved region of the vp72 gene.

TABLE 1
30 vp72 gene sequences of ASFV for primer design

| NCBI accession | |
|---|---|
| number | Gene size |
| MH713612.1 | 1,941 bp |
| AY578689.1 | 2,092 bp |
| AY578690.1 | 2,083 bp |
| AY578691.1 | 2,082 bp |
| AY578692.1 | 2,092 bp |
| AY578694.1 | 2,106 bp |
| AY578696.1 | 2,084 bp |
| AY578697.1 | 2,093 bp |
| AY578699.1 | 2,085 bp |
| AY578700.1 | 2,093 bp |
| AY578701.1 | 2,082 bp |
| AY578702.1 | 2,082 bp |
| AY578703.1 | 2,083 bp |
| AY578704.1 | 2,087 bp |
| AY578705.1 | 2,092 bp |
| AY578706.1 | 2,086 bp |
| AY578707.1 | 2,106 bp |
| AY578708.1 | 2,091 bp |
| KJ195685.1 | 1,941 bp |
| KT795353.1 | 1,941 bp |
| KT795356.1 | 1,941 bp |
| L76727.1 | 1,941 bp |
| MK554698.1 | 1,941 bp |
| MN207061.1 | 1,941 bp |
| MN793051.1 | 1,941 bp |
| MN886925.1 | 1,941 bp |
| MN886926.1 | 1,941 bp |
| MN886930.1 | 1,941 bp |
| MN886932.1 | 1,941 bp |
| MN886933.1 | 1,941 bp |

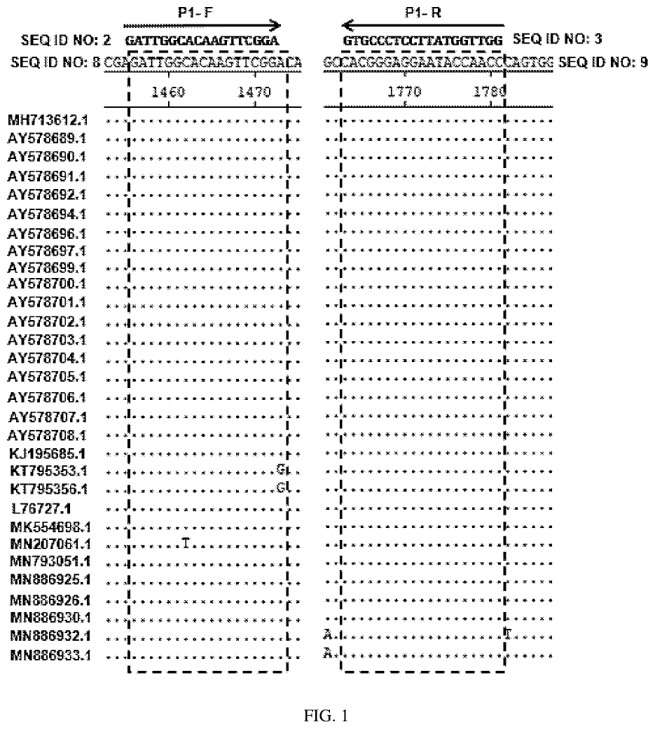

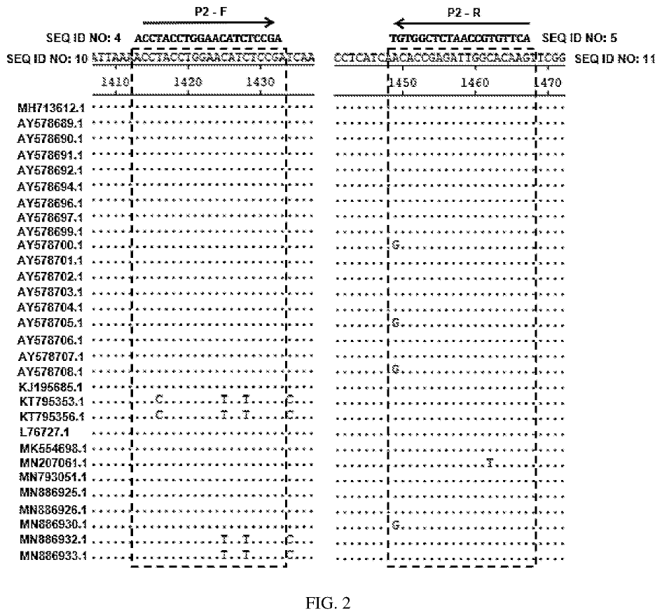

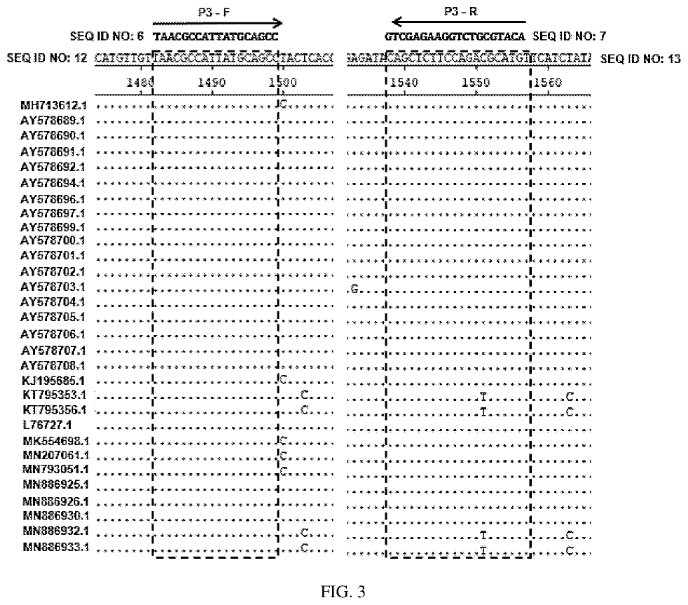

Primer information is shown in Table 2. P1 is a primer pair published in literature (Luo et al., 2016) that can specifically amplify the vp72 gene fragment of ASFV, and P2 and P3 are primer pairs designed and screened by the disclosure. The P1 primer pair is used as a control, which is more conservative, sensitive, and has a wider detection range compared to the PCR primer pair recommended by the World Organization for Animal Health (OIE). BGI Genomics Co., Ltd. is commissioned to synthesize primer pairs for P1, P2, and P3. The sequence alignment results show that, similar to the primer pair P1, the two pairs of primers (P2 and P3) designed by the disclosure are highly conserved among different ASFV strains (FIGS. 1-3).

TABLE 2
Specific primers for vp72 gene of ASFV

| Primer | Primer | Base | |
|---|---|---|---|
| pair | name | sequence | Location |
| P1 | vp72F-W | GATTGGCACAAGTT | 1456-1474 |
| | | CGGAC | |
| | | (SEQ ID NO: 2) | |
| | vp72R-W | GGTTGGTATTCCTC | 1763-1781 |
| | | CCGTG | |
| | | (SEQ ID NO: 3) | |
| P2 | vp72F-O | ACCTACCTGGAACA | 1413-1433 |
| | | TCTCCGA | |
| | | (SEQ ID NO: 4) | |
| | vp72R-O | ACTTGTGCCAATCT | 1449-1468 |
| | | CGGTGT | |
| | | (SEQ ID NO: 5) | |
| P3 | vp72F-O2 | TAACGCCATTATGC | 1482-1499 |
| | | AGCC | |
| | | (SEQ ID NO: 6) | |
| | vp72R-O2 | ACATGCGTCTGGAA | 1538-1557 |
| | | GAGCTG | |
| | | (SEQ ID NO: 7) | |

### Embodiment 2 Preparation of Plasmid Standards

1. Vector Construction

The Beijing Tsingke Xinye Biotechnology Co., Ltd. is commissioned to synthesize the conserved region of the nucleotide sequence shown in SEQ ID NO: 1 and insert the conserved region into the PUC57 plasmid vector to obtain a recombinant plasmid vector with a total length of 3135 bp, named PUC57-vp72.

2. Transformation

100 microliters (μL) Escherichia coli DH5a competent cells melted in ice bath are taken and added to a centrifuge tube including the recombinant plasmid vector PUC57-vp72, gently mixed, and then placed in ice bath for 30 minutes. After heat shock in 42° C. water bath for 60 seconds, the centrifuge tube is quickly transferred to ice bath for 2 minutes. 200 μL non-resistant sterile lysogeny broth (LB) culture medium, mixed evenly, and incubated in a shaker at 37° C. and 180 revolutions per minute (rpm) for 1 hour to allow cell recovery. 100 μL of bacterial solution is uniformly coated on LB agar medium containing 100 milligrams per liter (mg/L) ampicillin, the liquid on the surface of the medium is dried, and the medium is inverted and cultured at 37° C. overnight. On the next day, single colonies are selected and added to the LB liquid culture medium containing 100 mg/L ampicillin, and cultured in a shaker at 37° C. and 180 rpm for 10-16 hours. Then, the bacterial solution is sent to Beijing Tsingke Xinye Biotechnology Co., Ltd. for positive clone identification.

3. Plasmid Extraction

The positive clones with correct sequencing are performed with amplification culture, and then the PUC57-vp72 plasmid is extracted to obtain a plasmid standard stock solution. The copy number of plasmid standards is determined according to the following calculation formula: (ng/μL)×10⁻⁹×6.02×10²³/(bp×660)=copies/μL.

### Embodiment 3 Sensitivity Test of Fluorescence Quantitative PCR for Detection of ASFV

The plasmid standard stock solution prepared in the embodiment 2 is diluted in a 10-fold gradient to 1×10¹⁰ to 1×10¹ copies/μL as DNA templates. Using double-distilled water (ddH₂O) as a control, the primer pair P1 published in the literature and the primer pairs P2 and P3 designed by the disclosure (Table 2) are used to perform fluorescence quantitative PCR by TB Green dye method according to the following reaction system and conditions, and standard curves and dissolution curves are drawn.

The reaction system for fluorescence quantitative PCR is as follows.

| Reagent | Volume |
|---|---|
| TB Green Premix Ex Tag II (Tli Rnaseh Plus) (2×) | 5 μL |
| Upstream primer (10 μM) | 0.3 μL |
| Downstream primer (10 μM) | 0.3 μL |
| DNA template | 1 μL |
| ddH₂O without nuclease | Adding to 10 μL |

The reaction conditions for fluorescence quantitative PCR are: pre-denaturation at 95° C. for 30 seconds; denaturation at 95° C. for 5 seconds, 60° C. for 30 seconds, 40 cycles. The dissolution curve reaction condition is to gradually increase the temperature to 95° C.

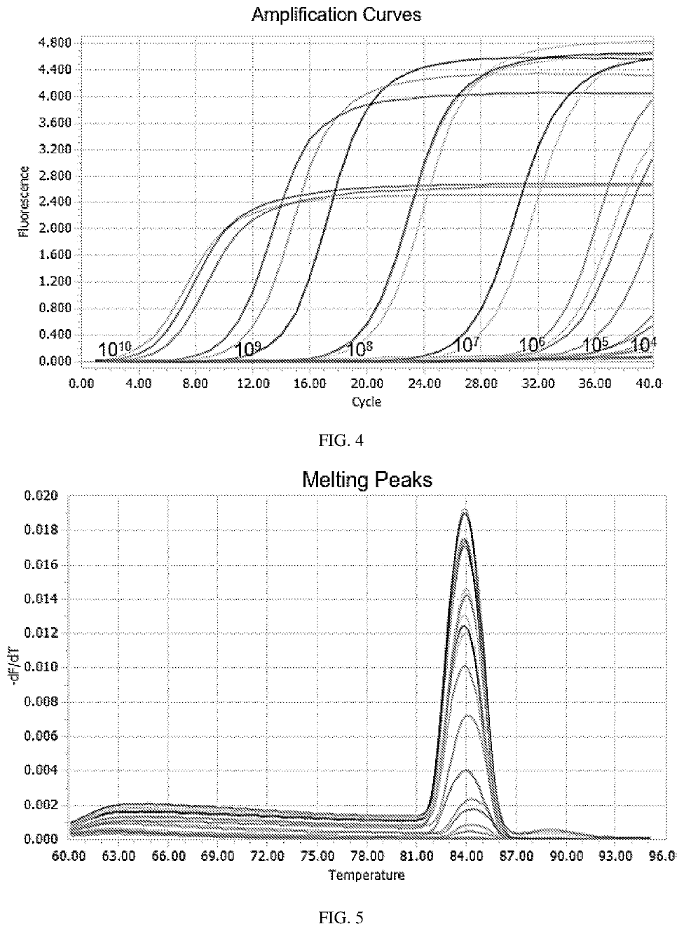

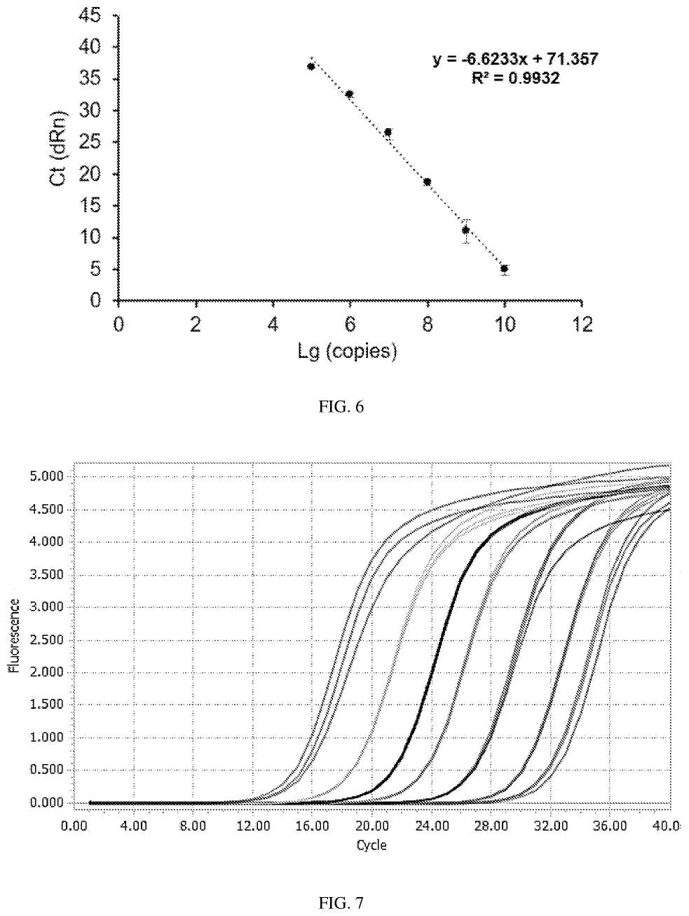

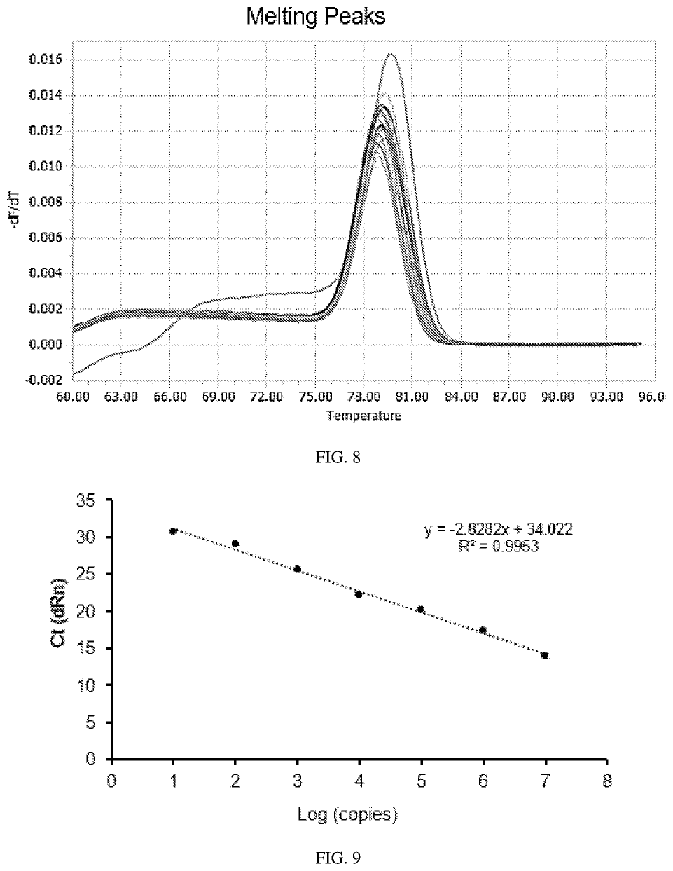

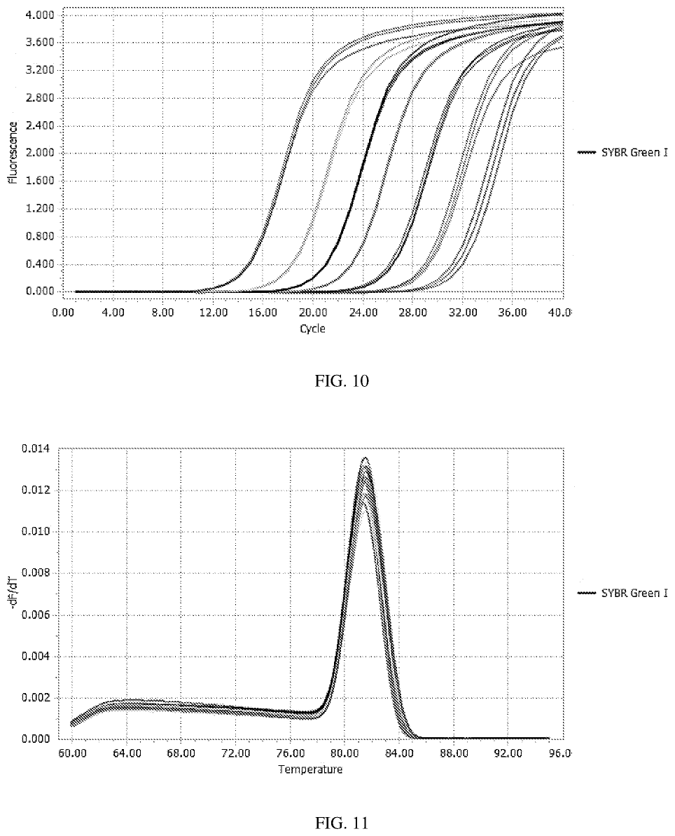

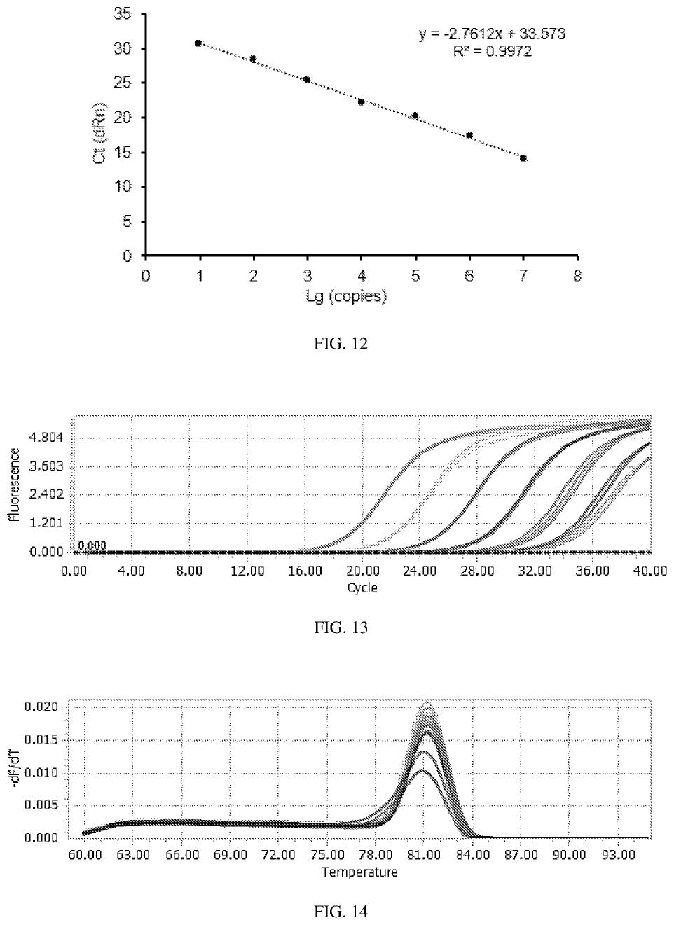

The results show that the amplification product of the P1 primer pair has no primer dimer, but the primer amplification efficiency is low, the correlation coefficient of the standard curve is 0.9932, and the lower limit of detection is 10⁴ copies (FIGS. 4-6). The amplification product of the P2 primer pair has no primer dimer, the correlation coefficient of standard curve is 0.9953, the lower limit of detection is 10 copies, the sensitivity is high, but the dissolution curve has biased peak, the specificity is poor (FIGS. 7-9). The amplification product of the P3 primer pair has no primer dimer, and the correlation coefficient of the standard curve is 0.9972, the lower limit of detection is 10 copies, the sensitivity is high, the peak shape of the dissolution curve is good, and the specificity is strong (FIGS. 10-12). Therefore, the P3 primer pair is the optimal primer pair for the detection of the vp72 gene of ASFV by fluorescence quantitative PCR.

### Embodiment 4 Repeatability Test of Fluorescence Quantitative PCR for Detection of ASFV

In order to further verify the specificity and stability of the ASFV fluorescence quantitative PCR detection method established by the disclosure, the plasmid DNA solution with dilution of 1×10² copies/μL in plasmid standards, genomic DNA of Bama Xiang pig, and ultrapure water are used as templates, the P2 and P3 primer pairs are used, and fluorescence quantitative PCR is performed through TB Green dye method according to the reaction system and conditions of fluorescence quantitative PCR in the embodiment 3. Three repeated measurements are set up for intra-assay and inter-assay reproducibility tests. A Ct value greater than 33 is considered a negative result, and the reproducibility of the method is evaluated using the coefficient of variation of the Ct value.

Genomic DNA of Bama Xiang pig is extracted from the blood of Bama Xiang pig using a blood genomic DNA extraction kit (TIANGEN Biotech (Beijing) Co., Ltd, Art. No. DP318-02) according to the procedures recorded in the kit introduction.

The results show that the amplification results of the P2 and P3 primer pairs have good reproducibility, and intra-assay and inter-assay coefficients of variation are less than 2% (Table 3). The amplification results using the genomic DNA of Bama Xiang pig or the ultrapure water as templates are negative. Therefore, the dye fluorescence quantitative PCR method based on the vp72 gene established by the disclosure has good reproducibility and specificity, and the detection results are stable and reliable.

TABLE 3
Specificity and reproducibility of the dye fluorescence quantitative PCR detection method

| | | | | | Coefficient | Inter-assay |
|---|---|---|---|---|---|---|
| Primer | | Repeat | Ct average | Ct standard | of variation | variation |
| pair | Template | number | value | deviation | C.V | C.V |
| P2 | Plasmid | 3 | 28.91 | 0.46 | 1.57% | 1.07% |
| | | 3 | 29.36 | 0.05 | 0.16% | |
| | | 3 | 28.78 | 0.42 | 1.46% | |
| | Pig DNA | 3 | — | — | — | — |
| | | 3 | — | — | — | |
| | | 3 | — | — | — | |
| | ddH₂O | 3 | — | — | — | — |
| | | 3 | — | — | — | |
| | | 3 | — | — | — | |
| P3 | Plasmid | 3 | 28.62 | 0.41 | 1.43% | 1.13% |
| | | 3 | 29.84 | 0.32 | 1.08% | |
| | | 3 | 28.80 | 0.26 | 0.90% | |
| | Pig DNA | 3 | — | — | — | — |
| | | 3 | — | — | — | |
| | | 3 | — | — | — | |
| | ddH₂O | 3 | — | — | — | — |
| | | 3 | — | — | — | |
| | | 3 | — | — | — | |

Note:
a Ct value greater than 33 is considered a negative result and is represented by “—”. “Plasmid” means that plasmid DNA at a dilution of 1 × 10² copies/μL in the plasmid standard is used as PCR template. “Pig DNA” means that the genomic DNA of Bama Xiang pig is used as the PCR template.

### Embodiment 5 Optimization of Fluorescence Quantitative PCR Conditions

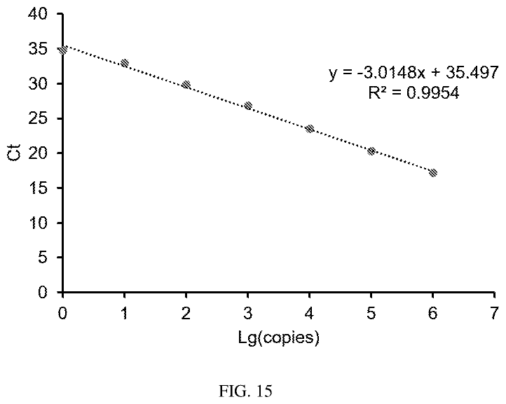

The plasmid standard stock solution prepared in the embodiment 2 is diluted in a 10-fold gradient to 1×10⁶ to 1×10⁰ copies/μL as DNA templates. The fluorescence quantitative PCR reaction system and reaction conditions for the P3 primer pair selected and designed by the disclosure are further optimized, and standard curves and dissolution curves are drawn. The optimal reaction system after optimization includes 10 μL of 2×TB Green Premix Ex Taq II (Tli RNaseH Plus), 0.8 μL of 10 micromoles per liter (μM) upstream primer of the P3 primer pair, 0.8 μL of 10 μM downstream primer of the P3 primer pair, 2 μL DNA Template, and ddH₂O without nuclease is added to make up to 20 μL. The optimal reaction conditions are as follows: step 1: pre-denaturation at 95° C. for 30 seconds; step 2: denaturation at 95° C. for 5 seconds, annealing and extension at 60° C. for 30 seconds, 40 cycles; step 3: dissolution curve reaction and gradually heated the temperature to 95° C. The amplification curves and dissolution curves are shown in FIGS. 13-15. The results of amplification using the P3 primer pair has good sensitivity, with a minimum detectable template concentration of 1 copy/μL.

### Embodiment 6 Specificity Test of Fluorescence Quantitative PCR for Detection of ASFV

Test materials are as follows.

Inactivated vaccine of pseudorabies virus (PRV) (Wuhan Keqian Biology Co., Ltd., approval number: veterinary drug name 170041044, the vaccine contains the PRV strains).

Inactivated vaccine of porcine circovirus type 2 (PCV2) (Wuhan Keqian Biology Co., Ltd., approval number: veterinary drug name 170041087, the vaccine contains porcine circovirus type 2, and the virus content per 2 mL before inactivation is ≥10^{7.0}TCID₅₀).

Inactivated vaccine of porcine parvovirus (PPV) (Wuhan Keqian Biology Co., Ltd., approval number: veterinary drug name 170041059, the vaccine contains PPV WH-1 strain, and the virus content per milliliter before inactivation is ≥10⁵-5TCID₅₀ or the blood coagulation titer is not less than 29).

Inactivated vaccine of H1N1 subtype of the swine influenza virus (also referred to as H1N1 influenza) (Wuhan Keqian Biology Co., Ltd., approval number: veterinary drug name 170041101, containing the H1N1 influenza TJ strain, and the virus content per 0.2 mL before inactivation is ≥10^{6.5}-EID₅₀).

Using the Mini BEST Viral RNA/DNA Extraction Kit Ver. 5.0 (TAKARA Bio INC., Art. No. 9766), the DNA of PRV, PCV2, PPV inactivated vaccines, and the RNA of the H1N1 subtype inactivated vaccine are extracted according to the kit instructions. Then, the RNA of the extracted H1N1 subtype inactivated vaccine is reverse transcribed using the PrimeScript™RT reagent Kit with gDNA Eraser (TAKARA Bio INC., Art. No. RR047A) according to the kit instructions, to obtain copy DNA (abbreviated as cDNA, also called complementary DNA).

Using the DNA of PRV, PCV2, PPV, and H1N1 cDNA as templates, while using 1.0×10⁵ copies/μL standard plasmid (PUC57-vp72) as the positive control, and ddH₂O as the negative control, the specificity is tested according to the optimal reaction system and conditions of fluorescence quantitative PCR in the embodiment 5. As shown in Table 4, the positive control (PUC57-vp72) shows specific amplification, while other viruses do not detect positive amplification, indicating that the method of the disclosure has good specificity.

TABLE 4
Specificity of TB Green fluorescence
quantitative PCR detection method

| Sample | Ct average value | Ct standard deviation |
|---|---|---|
| PUC57-vp72 | 20.84 | 0.12 |
| H1N1 | — | — |
| PCV2 | — | — |
| PPV | — | — |
| PRV | — | — |

Note:
a Ct value greater than 35 is considered a negative result, represented by “—”.

### Embodiment 7 Clinical Samples of ASFV Detected by Fluorescence Quantitative PCR

Four DNA samples of large white pigs infected with ASFV are used as positive samples, and the genomic DNA of Bama Xiang pigs is used as control samples, the detection is carried out according to the reaction system and reaction conditions of fluorescence quantitative PCR in the embodiment 3.

As shown in Table 5, there is no specific amplification curve in the four control samples, and the test results are negative. All four positive samples show specific amplification, and the test results are positive. Therefore, the coincidence rate of the fluorescence quantitative PCR detection method for ASFV vp72 gene established by the disclosure is 100%.

TABLE 5
Clinical sample detection results

| Sample | Ct average value | Ct standard deviation |
|---|---|---|
| Positive sample 1 | 14.46 | 0.01 |
| Positive sample 2 | 16.04 | 0.06 |
| Positive sample 3 | 16.97 | 0.01 |
| Positive sample 4 | 21.70 | 0.09 |
| Control sample 1 | — | — |
| Control sample 2 | — | — |
| Control sample 3 | — | — |
| Control sample 4 | — | — |

The above embodiments are merely some embodiments of the disclosure, not all embodiments. The above embodiments are merely used to explain and illustrate the technical solutions of the disclosure, and not to limit the scope of protection of the disclosure. Any modifications or changes made by those skilled in the art to the aforementioned embodiments within the scope of the art disclosed in the disclosure shall be included in the scope of protection of the disclosure.

Specifically, the related references of the disclosure are listed as follows.
Luo Y, Atim S A, Shao L, Ayebazibwe C, Sun Y, Liu Y, Ji S, Meng X Y, Li S, Li Y, et al. 2016. Development of an updated PCR assay for detection of African swine fever virus. Arch Virol 162(1): 191-199.
Malmquist W A, Hay D. 1960. Hemadsorption and cytopathic effect produced by African Swine Fever virus in swine bone marrow and buffy coat cultures. Am J Vet Res 21: 104-108.
Mebus C A. 1988. African swine fever. Adv Virus Res 35: 251-269.
Sánchez-Cordón PJ, Montoya M, Reis A L, Dixon L K. 2018. African swine fever: A re-emerging viral disease threatening the global pig industry. Vet J 233: 41-48.
Tulman E R, Rock D L. 2001. Novel virulence and host range genes of African swine fever virus. Curr Opin Microbiol 4(4): 456-461.
Wang N, Zhao D, Wang J, Zhang Y, Wang M, Gao Y, Li F, Wang J, Bu Z, Rao Z, et al. 2019. Architecture of African swine fever virus and implications for viral assembly. Science 366(6465): 640.
Wardley R C, Wilkinson P J. 1977. The association of African swine fever virus with blood components of infected pigs. Arch Virol 55(4): 327-334.
Zhao D, Liu R, Zhang X, Li F, Wang J, Zhang J, Liu X, Wang L, Zhang J, Wu X, et al. 2019. Replication and virulence in pigs of the first African swine fever virus isolated in China. Emerg Microbes Infect 8(1): 438-447.
Li Hongli, Cao Jinshan, Wang Junwei, et al. Establishment and application of real-time fluorescent quantitative PCR method for detection of African swine fever virus [J]. Chinese Journal of Animal Husbandry and Veterinary Medicine, 2012, 39 (006): 37-40.
Ren Ming, Niu Tingting, Yu Wanqi, et al. DEVELOPMENT OF TAQMAN PROBE REAL-TIME PCR METHOD FOR DETECTION OF AFRICAN SWINE FEVER VIRUS [J]. Chinese Journal of Animal Infectious Diseases, 2020028 (003): 42-48.
Zhang Quan, Zhu Hongfei, Sun Huaichang. Development of conventional PCR and Real-time PCR for detection of African swine fever virus [J]. Chinese Journal of Preventive Veterinary Medicine, 2007029 (006): 458-461.

What is claimed is:

1. Primers for detecting vp72 gene of African swine fever virus, wherein nucleotide sequences of the primers are as shown in SEQ ID NO: 6 and SEQ ID NO: 7.

2. A kit for detecting the vp72 gene of African swine fever virus, comprising the primers for detecting the vp72 gene of African swine fever virus as claimed in claim 1.

3. The kit as claimed in claim 2, comprising at least one of a positive control and a negative control.

4. The kit as claimed in claim 3, wherein the positive control is a standard plasmid containing a part or all of the vp72 gene, and the negative control is a liquid used to dilute the positive control.

5. The kit as claimed in claim 4, wherein the standard plasmid comprises a nucleotide sequence shown in SEQ ID NO: 1.

6. The kit as claimed in claim 5, wherein the positive control is a standard plasmid solution with a concentration of 1×10² copies per microliter (copies/μL).

7. The kit as claimed in claim 2, comprising a polymerase chain reaction (PCR) premix; wherein the PCR premix comprises a Taq enzyme, a deoxyribonucleotide triphosphate (dNTP) mixture, a TB Green dye, and a buffer.

8. A fluorescence quantitative PCR detection method for the vp72 gene of African swine fever virus, comprising:
performing dye fluorescence quantitative PCR on a genomic DNA of a sample to be detected by using the primers as claimed in claim 1.

9. The method as claimed in claim 8, wherein a reaction system of the dye fluorescence quantitative PCR comprises 5 μL of 2×TB Green Premix Ex Taq II (Tli RNaseH Plus), 0.3 μL of 10 micromoles per liter (μM) upstream primer, 0.3 μL of 10 μM downstream primer, 1 μL of DNA Template, and 3.4 μL of double-distilled water (ddH₂O) without nuclease.

10. The method as claimed in claim 8, wherein reaction conditions of the dye fluorescence quantitative PCR comprise: pre-denaturation at 95 Celsius degree (° C.) for 30 seconds, denaturation at 95° C. for 5 seconds, annealing and extension at 60° C. for 30 seconds, 40 cycles.

11. A fluorescence quantitative PCR detection method for the vp72 gene of African swine fever virus, comprising:
performing dye fluorescence quantitative PCR on a genomic DNA of a sample to be detected by using the kit as claimed in claim 2.

12. The method as claimed in claim 11, wherein a reaction system of the dye fluorescence quantitative PCR comprises 5 μL of 2×TB Green Premix Ex Taq II (Tli RNaseH Plus), 0.3 μL of 10 μM upstream primer, 0.3 μL of 10 μM downstream primer, 1 μL of DNA Template, and 3.4 μL of ddH₂O without nuclease.

13. The method as claimed in claim 11, wherein reaction conditions of the dye fluorescence quantitative PCR comprise: pre-denaturation at 95° C. for 30 seconds, denaturation at 95° C. for 5 seconds, annealing and extension at 60° C. for 30 seconds, 40 cycles.


SEQUENCE LISTING (13 sequences)

SEQ ID NO: 1 - DNA, 506 bp, synthetic construct
  source 1..506: mol_type = other DNA
tataggattaaaacctacctggaacatctccgatcaaaatcctcatcaacaccgagattggcacaagttcggacatgttgttaacgccattatgcagcctactcaccacgcagagataagctttcaggatagagatacagctcttccagacgcatgttcatctatatcggatattagccccgttacgtatccgatcacattacctattattaaaaacatttccgtaactgctcatggtatcaatcttatcgataagtttccatcaaagttctgcagctcttacatacccttccactacggaggcaatgcaattaaaacccccgatgatccgggtgcgatgatgattacctttgctttgaagccacgggaggaataccaacccagtggtcatattaacgtatccagagcaagagaattttatattagttgggacacggattacgtggggtctatcactacggctgatcttgtggtatcggcatctgctattaactttcttcttcttc

SEQ ID NO: 2 - DNA, 19 bp, synthetic construct
  source 1..19: mol_type = other DNA
gattggcacaagttcggac

SEQ ID NO: 3 - DNA, 19 bp, synthetic construct
  source 1..19: mol_type = other DNA
ggttggtattcctcccgtg

SEQ ID NO: 4 - DNA, 21 bp, synthetic construct
  source 1..21: mol_type = other DNA
acctacctggaacatctccga

SEQ ID NO: 5 - DNA, 20 bp, synthetic construct
  source 1..20: mol_type = other DNA
acttgtgccaatctcggtgt

SEQ ID NO: 6 - DNA, 18 bp, synthetic construct
  source 1..18: mol_type = other DNA
taacgccattatgcagcc

SEQ ID NO: 7 - DNA, 20 bp, synthetic construct
  source 1..20: mol_type = other DNA
acatgcgtctggaagagctg

SEQ ID NO: 8 - DNA, 23 bp, synthetic construct
  source 1..23: mol_type = other DNA
cgagattggcacaagttcggaca

SEQ ID NO: 9 - DNA, 27 bp, synthetic construct
  source 1..27: mol_type = other DNA
gccacgggaggaataccaacccagtgg

SEQ ID NO: 10 - DNA, 31 bp, synthetic construct
  source 1..31: mol_type = other DNA
attaaaacctacctggaacatctccgatcaa

SEQ ID NO: 11 - DNA, 32 bp, synthetic construct
  source 1..32: mol_type = other DNA
cctcatcaacaccgagattggcacaagttcgg

SEQ ID NO: 12 - DNA, 33 bp, synthetic construct
  source 1..33: mol_type = other DNA
catgttgttaacgccattatgcagcctactcac

SEQ ID NO: 13 - DNA, 33 bp, synthetic construct
  source 1..33: mol_type = other DNA
agatacagctcttccagacgcatgttcatctat